(19) United States
(12) Patent Application Publication
(10) Pub. No.: US 2010/0222798 A1
(43) Pub. Date: Sep. 2, 2010

(54) Method and apparatus for wound closure and for performing fasciotomies

(76) Inventor: Hector O. Pacheco, El Paso, TX (US)

Correspondence Address: NIXON & VANDERHYE, PC, 901 NORTH GLEBE ROAD, 11TH FLOOR, ARLINGTON, VA, 22203, US

(21) Appl. No.: 12/659,088

(22) Filed: Feb. 24, 2010

Related U.S. Application Data
(60) Provisional application No. 61/202,414, filed on Feb. 26, 2009.

(51) Int. Cl. A61B 17/32; A61B 17/04

(52) U.S. Cl. 606/180; 606/228; 606/167

(57) ABSTRACT

A method of closing an open wound in a body part, comprising making a plurality of spaced incisions in the skin adjacent to or near the wound sufficient to enable the skin to be stretched over the wound. Thereafter, the skin is stretched over the wound and secured in place over the wound to close it. Also, in accordance with a new method of decompression for fasciotomies, spaced incisions may be made in the skin overlying the muscle compartment to be decompressed to allow for adequate drainage and decompression of the swollen, tense and compromised muscle compartment, and preserve the integrity of the overlying skin to minimize or eliminate the need for skin grafting. A new and improved circular surgical scalpel may be used to make the spaced incisions in the skin.

### CROSS-REFERENCES TO RELATED APPLICATIONS

This application claims the priority of Provisional Patent Application No. 61/202,414 filed on Feb. 26, 2009.

### BACKGROUND OF THE INVENTION

1. Field of the Invention

The present invention relates to a new and improved method for facilitating wound closure and for performing fasciotomies and, more particularly, to such a method and apparatus wherein fenestrated incisions are made in the skin to facilitate wound closure and to perform fasciotomies.

2. Description of the Background Art

The management of wounds takes into account several components. For those wounds not amenable to reapproximation and closure because of a skin defect, wound edge retraction or similar compromise, the traditional surgical approach is to fill in the defect with a skin graft or rotational graft. The skin grafts can be split thickness, full thickness or free flaps. The rotational grafts can be adjacent fasciocutaneous grafts or advancement flaps with skin grafting of the resulting defect. Such treatments are time-consuming, complicated, costly and require long periods of healing and rehabilitation.

### BRIEF SUMMARY OF THE INVENTION

In accordance with the present invention, a bipedicle vascular full thickness skin graft is advanced to cover a wound skin defect. To accomplish this surgical advancement graft, multiple collinear incisions (i.e., fenestrations) substantially parallel to the wound edges are made to allow for viscoelasticity of the skin to stretch under appropriate tension for wound closure. This may be performed by a surgeon using a standard scalpel making, for example, one centimeter long incisions spaced about one centimeter apart. The intervening skin between adjacent incisions is left intact. The incisions are full thickness from the epidermis through the dermis and to the subcutaneous tissue. The underlying fascia is divided bluntly in line with the incisions to facilitate edema drainage and compliance of the skin. The skin is not undermined which allows for maximal vascularity through the adjacent skin tissue transfer. The skin is typically fenestrated adjacent to the wound edges but can also be fenestrated some distance away from the wound. The number of rows of incisions made in the skin is based on the amount needed for suitable closure of the wound without undue tension in the closing wound edges.

For muscle compartments demonstrating compartment syndrome (i.e., excessive intracompartment muscle pressure) either by objective evaluation or subjective clinical evaluation, a new method of decompression has been developed. In this new method of the present invention, the muscle compartment to be decompressed is identified and the overlying skin may be marked to allow for longitudinal multiple fenestrated incisions along the length of the compartment. The incisions can be made by a standard scalpel of any suitable type or by the use of the new circular scalpel described hereinafter.

To facilitate the above-described procedures, a new circular adjustable scalpel is used in accordance with the present invention which can make incisions either manually or mechanically to a desired depth and length based on a surgeon's preferences. The new circular adjustable scalpel provides a new and improved method of fenestrating the skin because the depth of the scalpel incision can be adjusted to allow for fenestration of the fascia and the overlying skin layers in a single operation rather than as separate steps as herein described.

The fenestration of the fascia by the circular scalpel or bluntly after the overlying skin has been fenestrated is a new method for performing fasciotomies of the underlying muscle compartments. The advantage of this method is that it allows for adequate drainage and decompression of the swollen, tense and compromised muscle compartment but preserves the integrity of the overlying skin to minimize or eliminate need for skin grafting.

### BRIEF DESCRIPTION OF THE DRAWINGS

FIG. 1 is a plan view of an open wound in the skin of a body part;

FIG. 2 is a plan view like FIG. 1 showing the open wound partially closed by sutures or the like to determine the stretching of the skin necessary to close the wound;

FIG. 3 is a plan view similar to FIG. 2 showing the skin area adjacent to the partially closed wound being marked for incisions in accordance with the present invention;

FIG. 4 is a plan view similar to FIG. 3 showing multiple incisions in the skin along the marked area to facilitate the stretching of the skin over the open wound;

FIG. 5 is a plan view similar to FIG. 4 showing the skin stretched over the wound as a result of the fenestrated incisions to fully close the wound by the application of sutures or the like;

FIG. 6 is a side elevational view, with parts broken away, of one side of a new and improved circular scalpel in accordance with the teachings of the present invention;

FIG. 7 is a plan view of the bottom of the circular scalpel shown in FIG. 6;

FIGS. 8a and 8b are partial plan views like FIG. 7 showing modified end bases;

FIG. 9 is a perspective view of the scalpel of FIG. 6 in position on the skin of a patient to form incisions therein;

FIG. 10 is a side elevational view like FIG. 6 showing a modified embodiment of the circular scalpel of the present invention; and

FIG. 11 is a side elevational view of a patient's leg showing the fenestrated incisions in the overlying skin for fasciotomy release in accordance with the present invention.

### DETAILED DESCRIPTION OF THE INVENTION

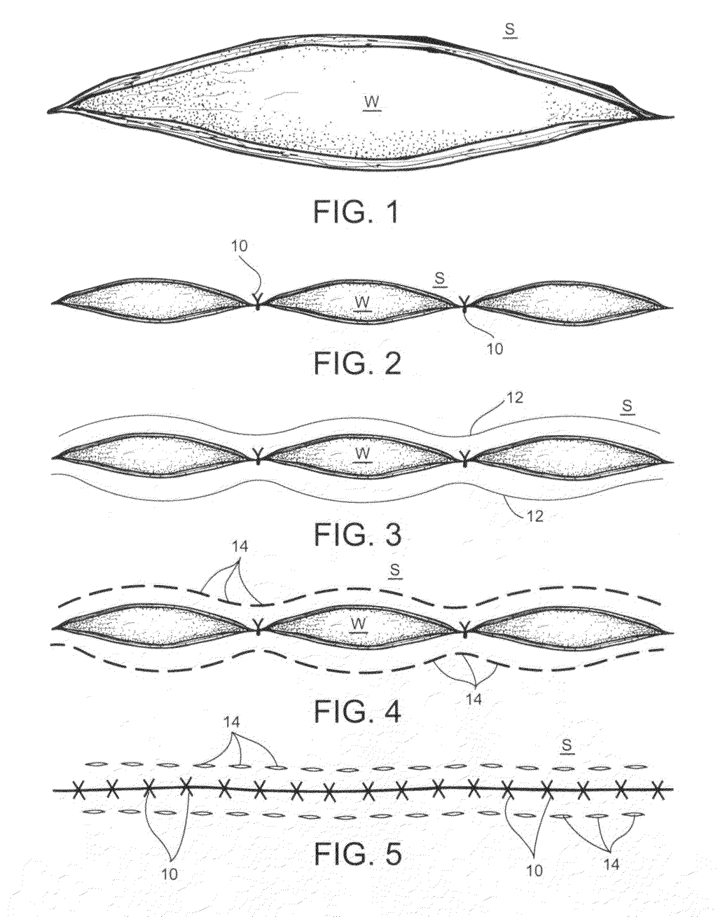

FIGS. 1-5 illustrate the new and improved method of the present invention for closing an open wound by enabling the skin surrounding the wound to be stretched over the wound to close it. First, the open wound W in the skin S of a body part, as shown in FIG. 1, is partially closed by sutures 10 or the like, as shown in FIG. 2, to determine the amount of stretching of the surrounding skin necessary to close the wound. The skin S surrounding the partially closed wound W may then be marked in areas 12 of the skin surrounding the partially closed wound W as shown in FIG. 3. The marking step is optional and may be omitted by a surgeon.

Thereafter, spaced incisions 14 are made in the marked or unmarked areas of the skin S to enable the skin surrounding the wound W to be stretched over the wound to close it. The number, length and size of the spaced incisions 14 will be determined by the amount of stretching of the skin necessary to close the wound W. The incisions 14 are made through the epidermis, dermis and to the subcutaneous tissue. The underlying fascia is bluntly divided in line with the incisions, or the incisions may be made through the fascia at the same time as through the epidermis, dermis and subcutaneous tissue with the use of the new circular scalpel to be described hereinafter. The fenestrated skin incisions 14 may be made through the skin S along the full length of the wound W or the incisions can be shorter or longer than the full length of the wound, depending on the stretching of the skin necessary to close the wound. Also, multiple rows of incisions may be made until adequate closure of the wound is obtained without undue skin tension. The skin may be fenestrated adjacent to the wound edges or may be fenestrated some distance away from the wound, if necessary. Any suitable size and/or spacing of the incisions may be used depending on the opinion of the surgeon.

As shown in FIG. 5, after the necessary incisions 14 have been made in the skin S, the skin is stretched over the wound W to close it with the use of sutures 10 or the like. In this manner, an open wound may be closed without the necessity of skin grafts or the like and in a simple procedure.

After the closing of the wound as shown in FIG. 5, the incisions 14 may be treated in any suitable manner to ensure drainage and healing. As an illustrative example, a sterile non-adherent dressing may be applied to the incisions 14 followed by a sterile gauze dressing or a Wound Vac sponge or the like. The incisions 14 usually heal in two to four weeks.

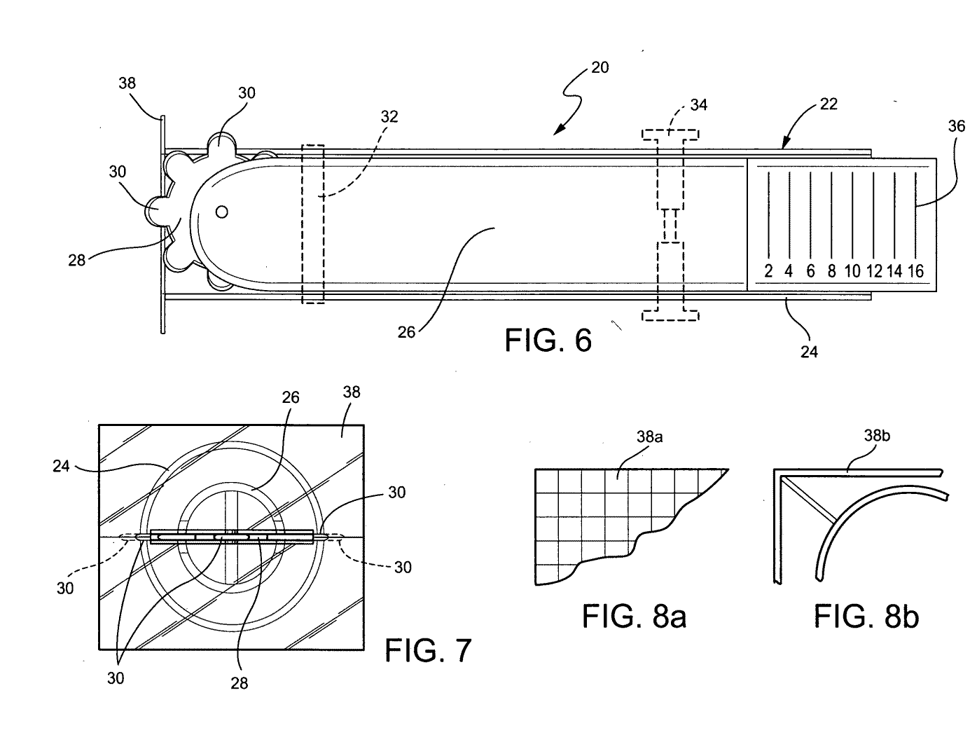

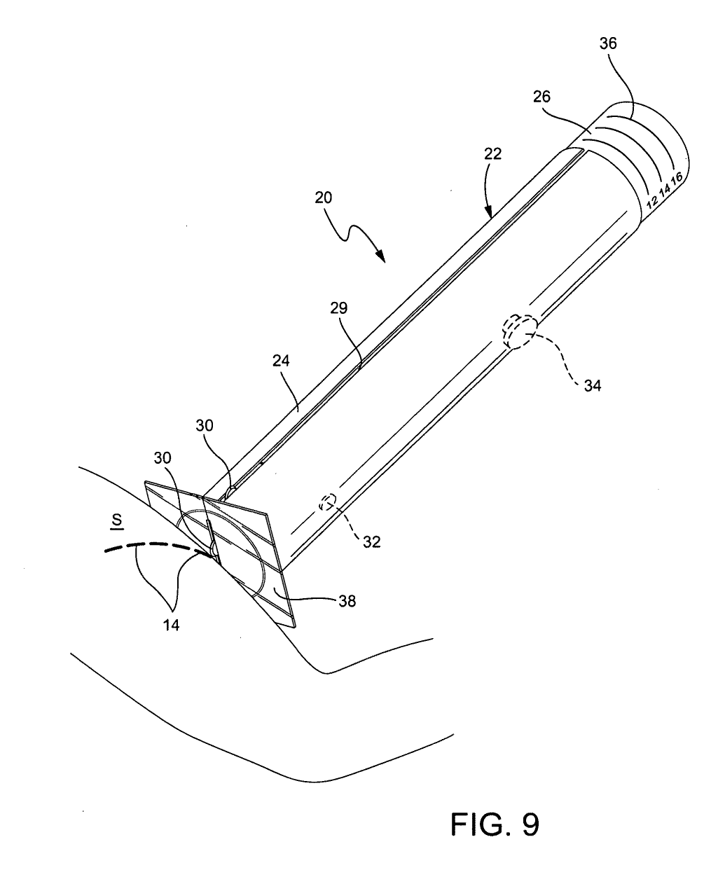

FIGS. 6-9 illustrate a new and improved apparatus in the form of a circular scalpel 20 for forming the fenestrated skin incisions shown in FIGS. 4 and 5 or for other purposes. The scalpel 20 comprises an elongated handle 22 having an outer housing 24 which may be cylindrical and an inner elongated section 26 slidably mounted in the outer housing 24. A circular cutter 28 is rotatably mounted on an inner end portion of the inner section 26 within the outer housing 24 and extends through an elongated slot 29 (FIG. 9) in one side of the outer housing 24 to enable the inner section 26 and the circular cutter 28 to be removed longitudinally from the outer end of the outer housing 24.

The circular cutter 28 has a plurality of spaced cutting teeth 30 on the periphery thereof which extend beyond the inner end of the outer housing 24 as shown in FIG. 6 when the circular cutter is rotated on the inner section 26. The cutting teeth 30 may be of any suitable size, shape and spacing, depending on the incisions to be made in the skin for closing an open wound or the like.

The handle 22 is provided with a first releasable locking device 32 of any suitable construction for engaging the circular cutter 28 to prevent its rotation on the inner section 26 during an initial incision to be made in the skin by a cutting tooth 30 extending outwardly from the inner end of the outer housing 24.

The handle 22 is provided with a second releasable locking device 34 of any suitable construction for removably retaining the inner section 26 in a desired longitudinal position relative to the outer housing 24. The outer end of the inner section 26 may be provided with indicia 36 visible at the outer end of the outer housing 24 to indicate the position of the inner section 26 relative to the housing 24 and the extent that the cutting teeth 30 extend beyond the inner end of the housing 24 for forming incisions in the skin.

The circular cutter 28 is removably mounted on the inner end portion of the inner section 26 so that it may be replaced by other circular cutters having different cutting teeth depths and configurations, if desired, when the inner section 26 is removed from the outer housing 24 by moving it longitudinally out of the outer end of the outer housing 24.

As shown in FIGS. 6-9, the inner end of the outer housing 24 is provided with a substantially transverse end base 38 through which the cutting teeth 30 extend. In the use of the scalpel 20 the inner base 38 thereof may be rested on the skin to be fenestrated, as shown in FIG. 9, and thereafter the scalpel 20 may be advanced along the skin to rotate the circular cutter 28 such that the cutting teeth 30 thereof make the desired spaced incisions 14 in the skin. To enable better viewing of the skin during the cutting operation, the base 38 may be transparent, may be in the form of a frame 38a or 38b with a grid or an open center portion, as shown in FIG. 8a or FIG. 8b respectively; or may be illuminated in any suitable manner.

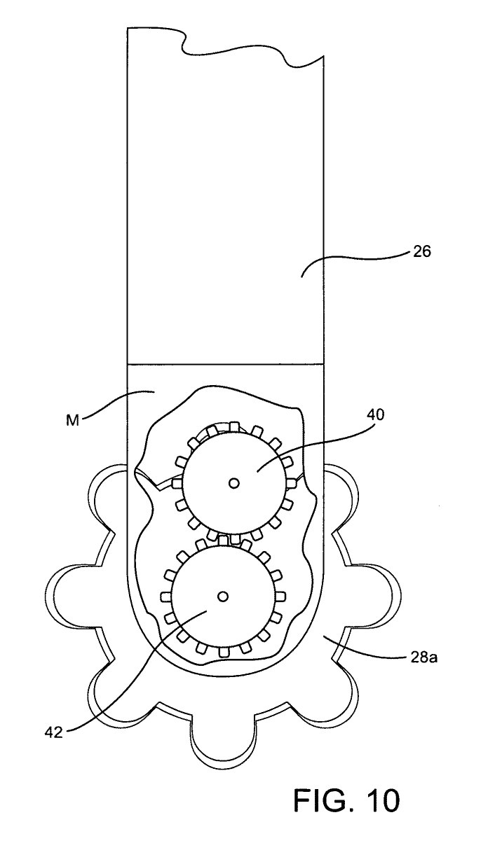

A modified embodiment of the scalpel 20 of FIGS. 6-9 is shown in FIG. 10 wherein the circular cutter 28a is rotatably driven by a motor M or the like mounted within the inner section 26 and operatively connected thereto such as by a gear 40 engaging a gear 42 on the cutter 28a.

Within the scope of the present invention, the inner section 26 of the handle 22 may be movably and removably mounted in the outer housing 24 in any suitable manner and the components of the handle 22 and cutter 28 may be of any suitable construction and may be formed of any suitable materials.

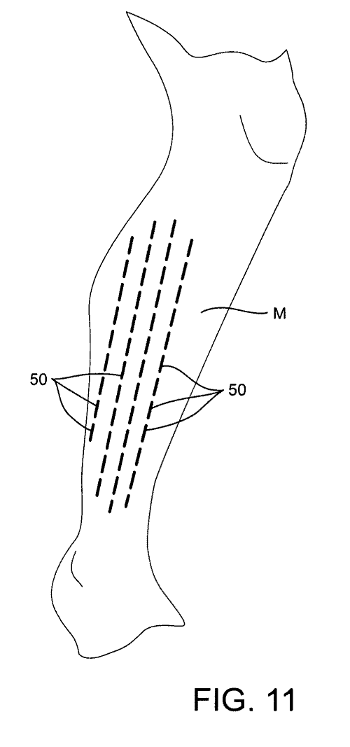

FIG. 11 illustrates a new method for performing fasciotomies of underlying muscle compartments to allow adequate drainage and decompression of the swollen, tense and compromised muscle compartment, and preserve the integrity of the overlying skin to minimize or eliminate the need for skin grafting or the like. In accordance with this method, the muscle compartment M to be decompressed is identified and a plurality of incisions 50 are made in the overlying skin along the length of the compartment M to allow for adequate drainage and decompression of the swollen, tense and compromised muscle compartment. The incisions 50 made in the skin enable the skin to stretch for adequate decompression. The nature of the incisions 50 may be the same as the incisions 14 for wound closure as shown in FIGS. 4 and 5 and described herein.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of closing an open wound in a body part, comprising:
making a plurality of spaced incisions in the skin adjacent to or near the wound sufficient to enable the skin to be stretched over the wound,
stretching the skin over the wound, and
securing the stretched skin in place over the wound to close the wound.

2. The method of claim 1 wherein the incisions are elongated and aligned so as to be substantially parallel to the edges of the wound.

3. The method of claim 2 wherein the incisions are on both sides of the wound.

4. The method of claim 2 wherein the incisions are on one side of the wound.

5. The method of claim 1 wherein the wound is partially closed before making the spaced incisions to provide an indication of the amount of stretching of the skin necessary to close the wound and thus the amount of incisions needed to enable the necessary stretching of the skin.

6. The method of claim 2 wherein the skin is marked to show the desired location of the incisions before they are made.

7. The method of claim 1 wherein the stretched skin is secured in place by sutures to close the wound.

8. The method of claim 3 wherein the incisions extend approximately the full length of each side of the wound.

9. The method of claim 1 wherein the incisions extend through the epidermis, dermis and to the subcutaneous tissue of the skin.

10. The method of claim 9 wherein the incisions extend through the underlying fascia.

11. The method of claim 3 wherein multiple rows of incisions are made that are in spaced, substantially parallel relation.

12. A method for fasciotomy release of excessive muscle compartment pressure, comprising:
identifying the skin area over the muscle compartment to be decompressed, and
making a plurality of incisions in the identified skin area sufficient to enable the skin to stretch and relieve the excessive muscle compartment pressure.

13. The method of claim 12 wherein the identified skin area is marked to show the desired location of the incisions before they are made.

14. The method of claim 12 wherein the incisions are elongated and aligned in the identified skin area.

15. The method of claim 14 wherein the incisions are in spaced rows in the identified skin area.

16. The method of claim 12 wherein the incisions extend through the epidermis, dermis and to the subcutaneous tissue of the skin.

17. The method of claim 16 wherein the incisions extend through the underlying fascia.

18. A scalpel for cutting spaced incisions in the skin, comprising:
an elongated handle; and
a circular cutter rotatably mounted on one end portion of said handle, said cutter having spaced cutting teeth on the periphery thereof for cutting spaced incisions in the skin when said cutter is rotatably moved in engagement with the skin by movement of said handle.

19. The scalpel of claim 18 further comprising a first locking device for locking said cutter in a selected position relative to said handle.

20. The scalpel of claim 18 wherein said handle comprises an outer elongated housing and an inner elongated section slidably mounted in said outer housing, said circular cutter being rotatably mounted on said inner section.

21. The scalpel of claim 20 further comprising a second locking device for locking said inner section in selected positions relative to said outer housing to vary the position of said cutter and the depth of incisions to be made by said cutting teeth when they engage the skin.

22. The scalpel of claim 21 wherein said inner section has indicia thereon adjacent the other end portion of the handle which indicate the position of said cutter relative to said outer housing and the extent to which said cutting teeth extend beyond said outer housing as a measure of the depth of the incisions to be made by said cutting teeth.

23. The scalpel of claim 22 wherein said outer housing at the one end portion of said handle has a substantially transverse end base with an opening through which said circular cutter extends, said base serving to support the outer housing on the skin for uniform pressure thereon when the circular cutter is moved along the skin.

24. The scalpel of claim 23 wherein said transverse end base is transparent to facilitate viewing of said circular cutter and the underlying skin when holding said outer housing of said handle near the other end portion thereof.

25. The scalpel of claim 23 wherein said transverse end base is a grid or an open frame to facilitate viewing of said circular cutter and the underlying skin when holding said outer housing of said handle near the other end portion thereof.

26. The scalpel of claim 18 wherein said circular cutter is removably mounted on said handle to facilitate the replacement of said circular cutter by another cutter of a different size and/or construction.

27. The scalpel of claim 20 wherein said outer housing comprises a longitudinal slot extending through the length thereof, and said circular cutter extends through said longitudinal slot to enable said inner section and said circular cutter to be removed longitudinally from said outer housing.

28. The scalpel of claim 27 wherein said circular cutter is removably mounted on an end portion of said inner section.

29. The scalpel of claim 18 further comprising a motor device operatively connected to said circular cutter for mechanically rotating said circular cutter on said handle.
